(19) United States
(12) Patent Application Publication
(10) Pub. No.: US 2023/0356000 A1
(43) Pub. Date: Nov. 9, 2023

(54) SYSTEM AND METHOD FOR LASER BASED ENDODONTIC TREATMENT

(71) Applicant: Convergent Dental, Inc., Needham, MA (US)

(72) Inventor: Charles Kerbage, Arlington, MA (US)

(21) Appl. No.: 18/143,848

(22) Filed: May 5, 2023

Related U.S. Application Data
(60) Provisional application No. 63/338,680, filed on May 5, 2022.

(51) Int. Cl. A61N 5/06

(52) U.S. Cl. CPC A61N 5/0624; A61N 5/0603; A61N 2005/0606; A61N 2005/0644

(57) ABSTRACT

The disclosed invention relates to a system and method for treatment of root canals, e.g., to clean, decontaminate and remove the smear layer. The system can include a laser source; a hand piece; and a device for directing radiation emitted by the laser source to a liquid creating pressure that result in irrigation. In some cases, the handpiece can include an optical element adapted to modulate a laser beam such that it is dispersive or focused.

### CROSS-REFERENCE TO RELATED APPLICATIONS

This application claims priority and benefit from U.S. Provisional Application No. 63/338,680, titled “System and Method for Laser Based Endodontic Treatment” and filed on May 5, 2022, which is hereby incorporated by reference herein in its entirety.

### FIELD OF THE INVENTION

The present invention generally relates to endodontic treatment and, more particularly, using a laser source, for decontamination, cleaning and debriding of root canals by directing energy pulses emitted by the laser source into a tooth filled with liquid such as irrigant.

### BACKGROUND OF THE INVENTION

Traditional endodontic techniques use mechanical instruments, ultrasonic or chemical irrigation in an attempt to clean and decontaminate the endodontic system in a tooth. Those techniques have not been demonstrated to be successfully removing all of the infective microorganisms and debris. This is because of the complex root canal anatomy and the inability of common irrigants to penetrate into the lateral canals and the apical ramifications (isthmus, fin a collateral root canals) where bacteria can often survive. It seems, therefore, appropriate to search for new materials, techniques and technologies that can improve the cleaning and decontamination of these anatomical areas.

Consequently, endodontists have focused on different irrigation techniques for the root canal in the recent past to seek the most effective chemical irrigation technique. Although conventional syringe techniques have been widely used for irrigating the root canal, it is still difficult to efficiently deliver the irrigant to the apical area, particularly in a narrow, curved canal. Accordingly, different agitation techniques have been proposed to improve the efficacy of irrigation solutions including hand agitation and sonic and ultrasonic devices. Studies have shown that the impact of alkaline solutions of NaOCl and EDTA in endodontics can be improved when these are agitated by ultrasonic source of energy or pulsed lasers. It creates fluid motion which improves the contact of the irrigant solutions with areas of the root canal walls that cannot be obtained by mechanical instruments. They also increase the temperature of these irrigants that results in better chemical actions on soft and hard tissues.

Other techniques using lasers to activate the irrigant solutions within the canal have been used for the purpose of disinfection and decontamination of the smear layer, bacteria and their byproducts within the root canal system. A major concern in root canal irrigation is the effective removal of the biofilm and of the smear layer, which is produced during root canal instrumentation and consists of norganic and organic material including bacteria and their byproducts. Laser activation of irrigants (LAI) resulted in a statistically more effective removal of debris and smear layer in root canals compared with traditional techniques and ultrasound. When LAI was first introduced it was believed that shock waves generated during the bubbles' collapse that would contribute to the efficacy of debridement and removal of the biofilm of organic tissue remains.

Studies with mid-infrared lasers, primarily absorbed by water, have reported the ability of debriding and cleaning the root canal walls superficially. The bacterial load reduction after erbium laser irradiation, for example, demonstrated mediocre effectiveness, because the wavelength low in depth of penetration and high absorption of the laser energy on the dentin surface. This is due to the fact that such lasers with wavelength in the near-infrared have negligible affinity for water and the hydroxyapatite of hard tissue that result in low effective results in debriding and cleansing the root canal surfaces and also caused characteristic morphological alterations of the dentinal wall. The smear layer was only partially removed, and the dentinal tubules primarily closed as a result of the melting of inorganic dentinal structures.

Folwaczny et al. evaluated the antibacterial effects of pulsed Nd:YAG laser irradiation at different energy settings in root canals without using photosensitizing dye and determined that laser radiation has anti-microbial effects in root canals. The results of a similar study by Piccolomini et al. showed an antibacterial effect of Di-odium Nd:YAG laser depending on the radiation frequency. An in vivo study evaluating the therapeutic effect of Nd:YAG laser in persistent lesions supported the use of laser, since it created an unfavorable environment for the continuing development of microorganisms. Gutknecht et al. investigated the antibacterial depth effect of the continuous wave of a 980-nm diode laser irradiation in bovine dentine, showing that laser can eliminate bacteria deep into the dentine.

In particular, photoacoustic technique called Photon-induced photoacoustic streaming (PIPS™) uses the Er:YAG (2940 nm) laser in combinations with EDTA solution equipped with a conical and stripped fiber tip. PIPS is based on the radial firing stripped tip with laser impulses of subablative energies of 20 mJ at 15 Hz for an average power of 0.3 W at 50 μs impulses. These impulses induce interaction of water molecules with peak powers of 400 W. This creates successive shock waves leading to formation of a powerful streaming of the antibacterial fluid located inside the canal, with no temperature rising With the PIPS technique, the fiber tip is held in the coronal aspect of the access preparation, and very short bursts of very low laser energy are directed down into the canal to stream irrigants throughout the entire root canal system. This technique results in much deeper irrigation than traditional methods (syringe, ultrasonic needle).

The effective absorption of the laser light by sodium hypochlorite leads to vaporization of the irrigating solution resulting in formation of vapor bubbles that causes secondary cavitation effects. In this procedure the Er:YAG laser creates photoacoustic shockwaves within the irrigant inside the root canal system. Perin et al. evaluated both Er:YAG laser (7 HZ, 100 mJ, 80 pulses/canal, 11 sec) and 1% NaOCl irrigations capacity against intra-canal microbiota and found its effectiveness to eliminate these microorganisms. Vezzani et al. evaluated the degree of dis-infection of the Er:YAG laser in root canals contaminated with five intracanal microorganisms at different frequencies and concluded that all the groups showed statistically similar results and no method totally eliminated microorganisms. Radatti et al. evaluated the efficacy of an Erbium,-Chromium:Yttrium,Scandium, Gallium, Garnet (Er,Cr:YSGG) laser powered hydrokinetic system (HKS) versus that of rotary instrumentation for root canal debridement. According to their results the debridement efficacy of the HKS with distilled water irrigation was unacceptable with 5.25 percent NaOCl irrigation and it was similar to that of rotary instrumentation. If the HKS was to be used for debridement, then NaOCl irrigation must be used for predictable tissue removal. Jha et al. stated both laser and rotary instrumentations are unable to eliminate root canal infections. Currently, great emphasis in terms of elimination of root canal infection is focused upon mechanical preparation and ultrasonic and laser activation methods in conjunction with using appropriate irrigation solutions requiring several steps and multiple devices that will require additional time. Additionally, procedures thus still rely on the use of ethylenediaminetetraacetic acid (EDTA) and sodium hypochlorite solutions, as mentioned, and are only partially effective in removing the smear layer and biofilm. Therefore, further optimization of laser-assisted irrigation and cleaning procedures is called for.

### REFERENCES

Ohl C-D, Arora M, Dijkink R, Janve V, Lohse D (2006) Surface cleaning from laser-induced cavitation bubbles. Appl Phys Lett 89(7):074102. https://doi.org/10.1063/1.2337506
Mir M, Gutknecht N, Poprawe R, Vanweersch L, Lampert F (2009) Visualising the procedures in the influence of water on the ablation of dental hard tissue with erbium:yttrium-aluminium-garnet and erbium, chromium:yttrium-scandium-gallium-gamet laser pulses. Lasers Med Sci 24(3):365-374. https://doi.org/10.1007/s10103-008-0571-1
Blanken J, De Moor R J, Meire M, Verdaasdonk R (2009) Laser induced explosive vapor and cavitation resulting in effective irrigation of the root canal. Part 1: a visualization study. Lasers Surg Med 41(7):514-519. https://doi.org/10.1002/lsm.20798
George R, Walsh L J (2008) Apical extrusion of root canal irrigants when using Er:YAG and Er,Cr:YSGG lasers with optical fibers: an in vitro dye study. J Endod 34(6):706-708. https://doi.org/10.1016/j.joen.2008.03.003
Blanken J W, Verdaasdonk R M (2007) Cavitation as a working mechanism of the Er, Cr:YSGG laser in endodontics: a visualization study. J Oral Laser Appl 7(2):97-106
De Moor R J, Blanken J, Meire M, Verdaasdonk R (2009) Laser induced explosive vapor and cavitation resulting in effective irrigation of the root canal. Part 2: evaluation of the efficacy. Lasers Surg Med 41(7):520-523. https://doi.org/10.1002/lsm.20797
Lukač N, Zadravec J, Gregorit P, Lukač M, Jezeršek M (2016) Wavelength dependence of photon-induced photoacoustic streaming technique for root canal irrigation. J Biomed Opt 21(7):075007-075007. https://doi.org/10.1117/1.JBO.21.7.075007
Peters O A, Bardsley S, Fong J, Pandher G, Divito E (2011) Disinfection of root canals with photon-initiated photoacoustic streaming. J Endod 37(7):1008-1012. https://doi.org/10.1016/j.joen.2011.03.016
Lloyd A, Uhles J P, Clement D J, Garcia-Godoy F (2014) Elimination of intracanal tissue and debris through a novel laser-activated system assessed using high-resolution micro-computed tomography: a pilot study. J Endod 40(4):584-587. https://doi. org/10.1016/j.joen.2013.10.040
Koch J D, Jaramillo D E, DiVito E, Peters O A (2016) Irrigant flow during photon-induced photoacoustic streaming (PIPS) using particle image velocimetry (PIV). Clin Oral Investig 20(2):381-386. https://doi.org/10.1007/s00784-015-1562-9
Deleu E, Meire M A, De Moor R J (2015) Efficacy of laser-based irrigant activation methods in removing debris from simulated root canal irregularities. Lasers Med Sci 30(2):831-835. https://doi.org/10.1007/s10103-013-1442-y
Olivi G, Divito E (2012) Photoacoustic endodontics using PIPS™: experimental background and clinical protocol. J Laser Health Acad 1:3
Kuhn K, Rudolph H, Luthardt R G, Stock K, Diebolder R, Hibst R (2013) Er:YAG laser activation of sodium hypochlorite for root canal soft tissue dissolution. Lasers Surg Med 45(5):339-344. https://doi.org/10.1002/lsm.22143
de Groot S D, Verhaagen B, Versluis M, Wu M K, Wesselink P R, van der Sluis L W (2009) Laser-activated irrigation within root canals: cleaning efficacy and flow visualization. Int Endod J 42(12): 1077-1083. https://doi.org/10.1111/j.1365-2591.2009.01634.x
Lukač N, Gregorit P, Jezeršek M (2016) Optodynamic phenomena during laser-activated irrigation within root canals. Int J
Thermophys 37(7):1-8. https://doi.org/10.1007/s10765-016-2071-z
Matsumoto H, Yoshimine Y, Akamine A (2011) Visualization of irrigant flow and cavitation induced by Er:YAG laser within a root canal model. J Endod 37(6):839-843. https://doi.org/10.1016j.joen.2011.02.035
Potocnik I (2008) Smear layer removal in endodontic treatment: materials and procedure. Zobozdrav Vestn 4-5:3
De Meyer S, Meire M A, Coenye T, De Moor R J (2017) Effect of laser-activated irrigation on biofilms in artificial root canals. Int Endod J 50(5):472-479. https://doi.org/10.1111/iej.12643
Cheng X, Tian T, Tian Y, Xiang D, Qiu J, Liu X, Yu Q (2017) Erbium:yttrium aluminum garnet laser-activated sodium hypochlorite irrigation: a promising procedure for minimally invasive endodontics. Photomed Laser Surg 35(12):695-701. https://doi.org/10.1089/pho.2017.4274
Verstraeten J, Jacquet W, De Moor R J G, Meire M A (2017) Hard tissue debris removal from the mesial root canal system of mandibular molars with ultrasonically and laser-activated irrigation: a micro-computed tomography study. Lasers Med Sci 32(9):1965-1970. https://doi.org/10.1007/s10103-017-2297-4
DiVito E, Peters O A, Olivi G (2012) Effectiveness of the erbium: YAG laser and new design radial and stripped tips in removing the smear layer after root canal instrumentation. Lasers Med Sci 27(2): 273-280. https://doi.org/10.1007/s10103-010-0858-x
Violich D R, Chandler N P (2010) The smear layer in endodontics—a review. Int Endod J 43(1):2-15. https://doi.org/10.1111/j.1365-2591.2009.01627.x

### SUMMARY OF THE INVENTION

In view of the foregoing, it is desirable to provide a laser based treatment device, system, and/or method that provides laser agitation and/or cavitation of irrigants in endodontic canals (or root canals). In general, the present invention is directed toward a handpiece that is configured to apply a laser irradiation of desired parameters to maximize efficiency and efficacy of irrigation in endodontic canals. In some embodiments, the laser based system and method as described herein may effectively clean root canals using a single device and in a short treatment time without weakening the structure of the teeth. In some embodiments, the present invention provides a dental irrigation system, including a laser and a liquid, configured to cutting hard tissue (the top part of tooth) and providing laser energy into the liquid for debriding, cleaning, and/or disinfecting of root canals.

In one aspect, the invention relates to a system for providing a laser treatment to an endodontic canal to decontaminate, clean, and remove a smear layer of the endodontic canal. The system can include a handpiece including a CO₂ laser source for generating and delivering a plurality of laser pulses of a laser beam having a wavelength in a range from 9 μm to 11 μm; and an optical element to adapt the laser beam such that the plurality of laser pulses are delivered into a treatment site at the endodontic canal, wherein the laser pulses includes a laser irradiation energy level, and wherein the laser irradiation energy level creates a pressure wave and induces agitation or cavitation of irrigants in the root canal.

In some embodiments, the laser treatment provides a rate of irrigation or movement of irrigants from about 1 to about 20 mm/s.

In some embodiments, the laser treatment provides a substantially complete removal of the smear layer.

In some embodiments, the laser irradiation energy level of a laser pulse of the plurality of laser pulses is no more than about 1 J/cm².

In some embodiments, a laser pulse of the plurality of laser pulses includes a duration from about 1 to about 100 μsec.

In some embodiments, the system further includes a beam guidance system, wherein the beam guidance system is adapted to direct the plurality of laser pulses to respective tissue locations in a pattern. In some instances, the pattern includes a number of locations from about 15 locations to about 1500 locations. In some instances, the beam guidance system is adapted to repeat directing the plurality of laser pulses to respective tissue locations in a pattern. In some instances, the pattern includes at least one tissue location, at least one location non-adjacent to the tissue location, and at least one location adjacent to the tissue location.

In some embodiments, the handpiece is adapted to form an exit orifice and operatively connected to the beam guidance system for delivering the laser beam to the hard treatment area. In some instances, the handpiece further includes a focusing optic and at least one optical lens, wherein the at least one optical lens is disposed between the beam guidance system and a tip. In some instances, the at least one lens comprises two lenses. In some instances, the focusing optic and the at least one lens are configured to increase a diameter of the laser beam. In some instances, the focusing optic and the at least one lens are configured to generate a collimated laser beam.

In another aspect, the invention relates to a method for treating a treatment area of hard tissue, the method comprising the steps of: generating a plurality of laser pulses of a laser beam having a wavelength from about 9 μm to about 10 μm using a CO₂ laser source; and directing the plurality of laser pulses to respective tissue locations within a treatment area.

In some embodiments, the laser source operates in the range of 9-11 μm wavelength, such as a CO₂ laser. In general, the system uses a laser source to create a pulsed laser beam to access the treatment region of the tooth. In some embodiments, the present invention features a handpiece and an add on tip for directing radiation (e.g., a laser beam) in the near- to far-infrared spectra (e.g., 9-11 μm wavelength range), to the treatment area and a tip attached to the handpiece and is in contact with the liquid to deliver effective energy to the liquid.

In some embodiments, the tip comprises a hollow tube that is attached to the handpiece combined with an optical insert. Such configuration can provide two main advantages. First, the optical insert can be designed to have specific radius of curvature to provide desired depth of focus and size of the laser beam so at to maximize efficiency of energy transfer from the laser beam to the irrigant. Secondly, the optical insert acts as a stopper or a seal to the tube so that the liquid does not get pulled into the tip by means of capillary force and results in unfavorable effect of attenuating the laser beam.

In one aspect, embodiments of the invention feature a system for delivering the liquid into the root canal through a tube embedded in the handpiece. The system can include a CO₂ laser source for generating a plurality of laser pulses of a laser beam having a wavelength in a range from 9 μm to 11 μm, and a beam guidance system for directing the plurality of laser pulses to the hard tissue.

In various embodiments, each laser pulse has a fluence of a range between 0.01-1 J/cm² and/or a duty cycle in a range from 0.1 to 5 percent. The beam guidance system can direct the plurality of laser pulses to respective tissue locations in a pattern. The pattern can have a number of locations (e.g., 15 to 1500 locations or 30 to 217 locations). The total pattern time can be in a range from 0.001 to 0.5 seconds.

In some cases, the pattern includes a first tissue location, at least one location non-adjacent to the first tissue location, and a location adjacent to the first tissue location.

In various embodiments, the system can also include a handpiece forming an exit orifice and operatively connected to the beam guidance system for delivering the laser beam to the treated area. In some cases, the exit orifice of the tip can direct the laser beam toward the area alongside cooling fluid (such as air/mist) and a fluoride treatment. The handpiece can also include a focusing optic (insert) disposed between the beam guidance system and the exit orifice.

In different embodiments, the optics (insert) is designed to deliver the laser energy inside the liquid by focusing of the laser beam (e.g., a focused laser beam) at certain distances inside the fluid. In some instances, the laser beam is focused at a range inside the liquid (a range from 0.1-15 mm).

Another advantage of the optic insert is that it provides a seal of the tip, which is hollow, and does not allow the fluid to get into the the tip by means of capillary force and attenuate the laser energy.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the invention. In the following description, various embodiments of the present invention are described with reference to the following drawings, in which:

Figure (FIG.) 1 is a side schematic view of a laser based treatment system, including a handpiece, a consumable tip and an optical insert, according to various embodiments.

FIG. 2 is a side cross-sectional schematic view of a handpiece, according to various embodiments.

FIG. 3 is a side view providing an example laser treatment, during which the bubble is created in a sealed root canal phantom filled with liquid, according to various embodiments.

FIG. 4 is a view providing example of ray tracing modeling of the laser beam focused by the optical insert 3, according to various embodiments.

FIG. 5 is a chart providing example laser treatment and operation parameter values, according to various embodiments.

### DETAILED DESCRIPTION

Various embodiments of the present invention are directed to an improved laser treatment device that overcomes the shortcomings of conventional methods for root canal disinfection. The device can include a hand piece with a special tip that delivers (i) laser pulses that induces bubbles in the fluid to remove the smear layer and (ii) a laser beam having a focused beam, (iii) liquid (e.g., saline, hydrogen peroxide) to an oral treatment region and (iv) delivery system for the irrigant.

FIG. 1 is a side schematic view of a laser based treatment system, according to various embodiments. In some embodiments, the laser based treatment system includes a handpiece 1, an optical element (e.g., a consumable tip 2 and/or an optical lens), and an optical insert 3. In some embodiments, the handpiece 1 includes a laser source to generate a plurality of laser pulses of a laser beam (e.g., a focused laser beam). For some applications, as described herein, a laser source (e.g., a CO₂ laser source) operating at a wavelength in a range of 9-11 μm (e.g., 9.3 μAm), is desirable for such treatments.

In some embodiments, the handpiece 1 includes at least a focusing optic and at least one optical lens (e.g., 1, 2, 3, 4, 5 or more lens), where the optical lens is disposed between the beam guidance system and a tip (e.g., the consumable tip 2). When the laser based system is in operation, the focusing optic and the optical lens are configured to adapt (e.g., increase or decrease) a diameter of the laser beam and/or to generate a collimated laser beam.

In some embodiments, the delivery of the laser beam is achieved by the handpiece 1, which may be structured and designed to receive the optical element, e.g., the consumable tip 2. In some embodiments, the plurality of laser pulses include at least one laser irradiation energy level (e.g., fluence), and the laser irradiation energy level (e.g., fluence) creates a pressure wave and induces agitation or cavitation of irrigants in the root canal. In some embodiments, the laser irradiation energy level of a laser pulse of the plurality of laser pulses is no more than about 0.1, 0.2, 0.3, 0.4, 0.5, 0.6, 0.7, 0.8, 0.9, 1, 2, 3, 4, or 5 J/cm². In preferred embodiments, the laser irradiation energy level of a laser pulse of the plurality of laser pulses is no more than about 1 J/cm². In some embodiments, a laser pulse of the plurality of laser pulses includes a duration from about 0.1 μsec to about 1000 μsec, from about 0.2 μsec to about 500 μsec, from about 0.5 μsec to about 500 μsec, from about 1 μsec to about 500 μsec, from about 1 μsec to about 200 μsec, or from about μsec 1 to about 100 μsec. In preferred embodiments, a laser pulse of the plurality of laser pulses includes a duration from about 1 μsec to about 100 μsec.

In some embodiments, the optical element is configured to adapt the laser beam such that the plurality of laser pulses are delivered into a treatment site at the endodontic canal. For example, the consumable tip 2 can include at least one optical lens (or optical insert) 3 to modulate a laser beam passing therethrough. The ability to replace or remove the consumable tip 2 allows switching the laser between treatment modes, such as an ablative mode to a non-ablative mode and vice versa.

In some embodiments, the optical insert 3 provides: (i) seal to the tip such that no capillary force pulls the liquid into the tip and/or (ii) flexibility in obtaining a focusing beam at different locations and laser energy levels to provide optimal treatment efficacy. The laser irradiation energy level (e.g., fluence) can create a pressure wave and induces agitation (or cavitation) of irrigants located within endodontic canals.

In addition, the laser based treatment system may further include a tubing (or tube opening) 4 in the handpiece 1 throughout the consumable tip 2 to deliver cooling fluids (e.g., air, water, hydrogen peroxide and combinations thereof) to provide fluoride based fluid or a hydrogen based gel ahead of or during the treatment, as shown in FIG. 1.

In certain embodiments, the CO₂ laser is accompanied with a marking beam (e.g., green in color) that serves as a guidance of the location of the laser beam on the target tissue. In other embodiments, the irradiation of the laser comprises a pattern.

In some embodiments, the laser based treatment system provides a laser treatment to an endodontic canal to decontaminate, clean, and/or remove at least part of a smear layer of the endodontic canal. In some embodiments, the laser treatment provides a rate of irrigation or movement of irrigants from about 1 millimeters per second (mm/s) to about 20 mm/s. In some embodiments, the laser treatment provides tissue dissolution of the smear layer. In some embodiments, the laser treatment provides a substantially complete removal of the smear layer.

In some embodiments, the laser based treatment system further includes a beam guidance system disposed in the handpiece 1. In some embodiments, the handpiece 1 forms an exit orifice and is operatively connected to the beam guidance system for delivering the laser beam to a treatment area (e.g., of a hard tissue).

In some embodiments, the beam guidance system is adapted to direct (and/or repeat directing) the plurality of laser pulses to respective tissue locations in a pattern that includes a plurality of locations. In some embodiments, the pattern includes a plurality of locations including about 1 location to about 5000 locations, about 5 locations to about 5000 locations, about 10 locations to about 2000 locations, or about from about 15 locations to about 1500 locations. In preferred embodiments, the pattern includes a plurality of locations including about 15 locations to about 1500 locations. In some embodiments, the plurality of locations includes at least one tissue location, at least one location non-adjacent to the tissue location, and/or at least one location adjacent to the tissue location.

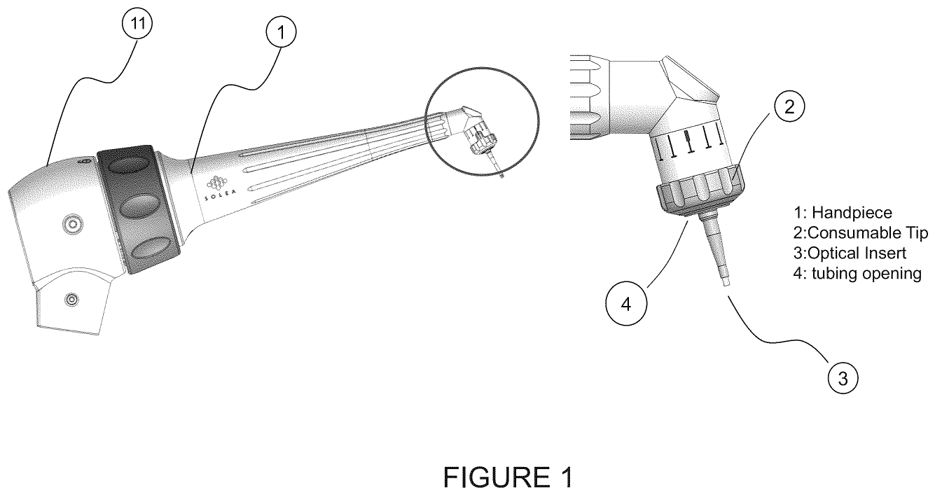

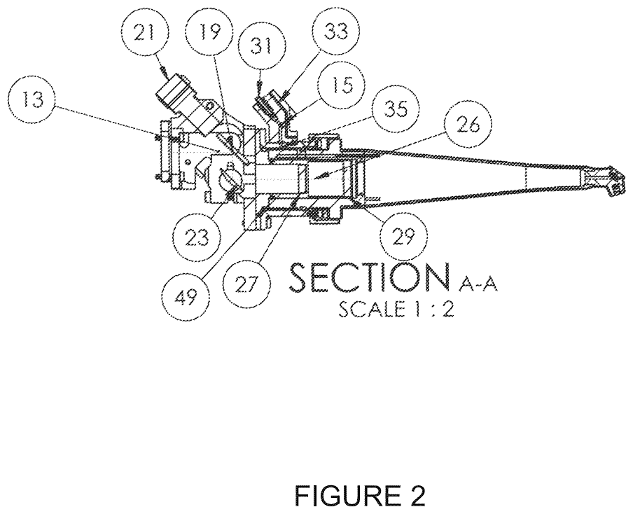

FIG. 2 is a side cross-sectional schematic view of a handpiece and a main chamber, according to various embodiments. Examples of such handpiece and associated components are described in U.S. Pat. Nos. 9,622,833 and 10,182,881, which are incorporated herein by reference in their entireties.

With reference to FIG. 2, a main chamber 11 (FIG. 1) includes a main optical subsystem 13 and a primary fluid supply system 15 affixed to the hand piece 1. In one embodiment, the optical subsystem includes an articulating arm (not shown) through which a laser beam exits toward a first galvanometer mirror 19. The first galvanometer mirror 19 can be attached to a shaft of a first galvanometer 21. The angular orientation in a first axis of the first galvanometer mirror 19 and, therefore, the laser's angle of incidence onto the first galvanometer mirror 19 relative to the first axis is servo-mechanically controlled by the first galvanometer 21. The first galvanometer mirror 19 is generally orientated so that the beam once reflected off the first galvanometer mirror is directed toward a second galvanometer mirror 23, which is attached to a shaft of a second galvanometer 25. The angular orientation in a second axis of the second galvanometer mirror 23 and, therefore, the laser's angle of incidence onto the second galvanometer mirror 23 relative to the second axis is servo-mechanically controlled by the second galvanometer 25. The second galvanometer mirror 23 is generally oriented so that the beam once reflected off the second galvanometer mirror 23 is directed along an optical axis 26, toward and through a first focusing optic 27 that is generally centered along the optical axis 26. The first focusing optic 27 generally has a concave curvature. In some embodiments, the first focusing optic 27 defocuses the beam, increasing the beam width as the beam is directed toward and through a second focusing optic 29 that is also generally centered around the optical axis 26. The second focusing optic 29 can have a generally convex curvature and may be larger in diameter than the first focusing optic 27 to allow for the increased beam width. The curvatures and locations of the first and second focusing optics 27 and 29 can be selected such that the beam is focused outside the hand piece at a selectable distance from an orifice thereof.

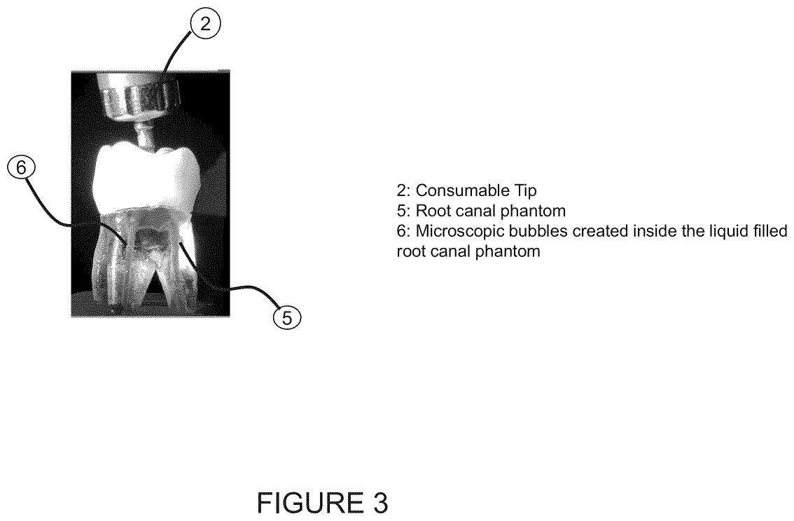

FIG. 3 is a side view providing an example laser treatment, during which the bubble is created in a sealed root canal phantom filled with liquid, according to various embodiments. In some embodiments, the laser treatment is a bench top testing using a prototype handpiece for creating bubble and fluid movement inside a mockup of a root canal.

As shown in FIG. 3, during a laser treatment as described herein, a laser beam (e.g., a laser beam created by a handpiece 1) can pass through the consumable tip 2 and be focused by the optical inset 3 to provide laser energy to a liquid filled endodontic canal (or root canal phantom 5). The laser irradiation energy level (fluence) can create a pressure wave and induce agitation (or cavitation) of liquid (e.g., irrigants including ethylenediaminetetraacetic acid (EDTA) and/or sodium hypochlorite in water solutions) located within the endodontic canal by generating microscopic bubbles 6 so as to achieve decontamination, cleaning, debriding, and/or disinfecting of the root canal.

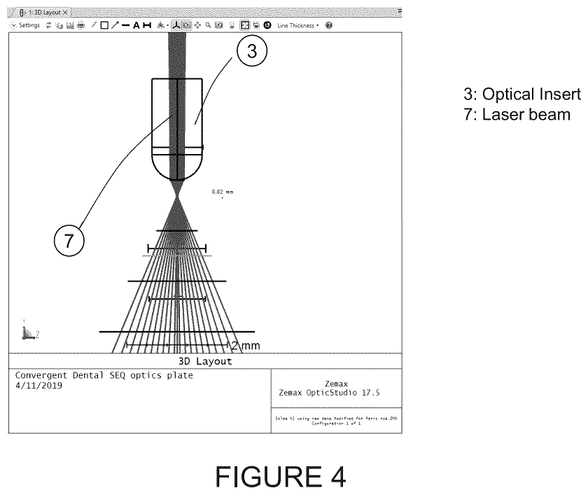

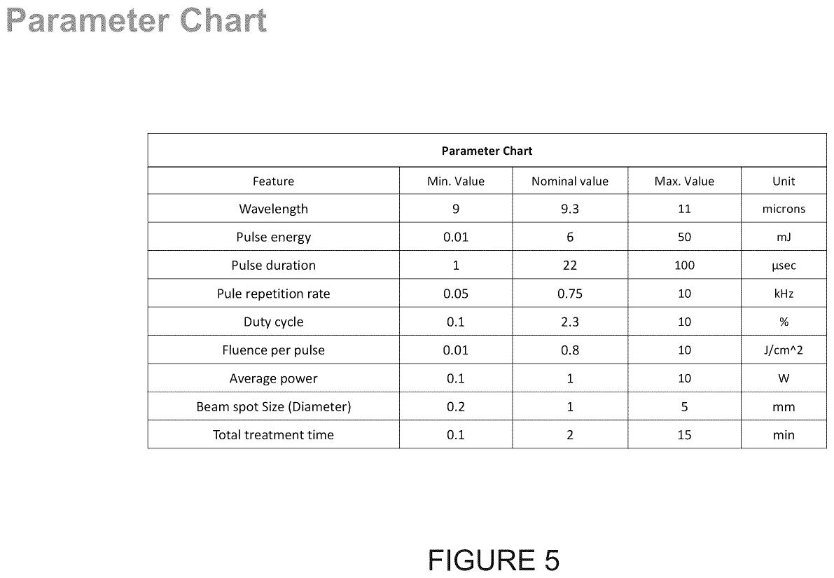

FIG. 4 is a view providing example of ray tracing modeling of the laser beam focused by the optical insert 3, according to various embodiments. For example, FIG. 4 is a simulation of an optimized laser beam being focused by the optical inset to provide optimal efficacy of laser energy transfer inside the liquid (e.g., an irrigant). In some embodiments, the laser treatment and/or operation parameters used in the simulation as shown in FIG. 4 are included in FIG. 5, as described in detail herein.

FIG. 5 is a chart including example laser treatment and operation parameters, according to various embodiments. Laser parameters (e.g., power, repetition rate, pulse duration, and laser beam overlap) may be configured to have an optimal outcome efficiency to remove carbonate without damaging the material (i.e., optical cartridge or lens) itself. In some embodiments, the laser source may be spatially scanned to provide different pulse energy at different locations of a root canal, as will be appreciated by those skilled in the art.

Each numerical value presented herein is contemplated to represent a minimum value or a maximum value in a range for a corresponding parameter. Accordingly, when added to the claims, the numerical value provides express support for claiming the range, which may lie above or below the numerical value, in accordance with the teachings herein. Every value between the minimum value and the maximum value within each numerical range presented herein (including in the chart shown in FIG. 5), is contemplated and expressly supported herein, subject to the number of significant digits expressed in each particular range.

Having described herein illustrative embodiments of the present invention, persons of ordinary skill in the art will appreciate various other features and advantages of the invention apart from those specifically described above. It should therefore be understood that the foregoing is only illustrative of the principles of the invention, and that various modifications and additions can be made by those skilled in the art without departing from the spirit and scope of the invention. Accordingly, the appended claims shall not be limited by the particular features that have been shown and described but shall be construed also to cover any obvious modifications and equivalents thereof.

What is claimed is:

1. A system for providing a laser treatment to an endodontic canal to decontaminate, clean, and remove a smear layer of the endodontic canal, the system comprising:
a handpiece comprising a CO₂ laser source for generating and delivering a plurality of laser pulses of a laser beam having a wavelength in a range from 9 μm to 11 μm; and
an optical element to adapt the laser beam such that the plurality of laser pulses are delivered into a treatment site at the endodontic canal,
wherein the laser pulses includes a laser irradiation energy level, and
wherein the laser irradiation energy level creates a pressure wave and induces agitation or cavitation of irrigants in the root canal.

2. The system of claim 1, wherein the laser treatment provides a rate of irrigation or movement of irrigants from about 1 to about 20 mm/s.

3. The system of claim 1, wherein the laser treatment provides a substantially complete removal of the smear layer.

4. The system of claim 1, wherein the laser irradiation energy level of a laser pulse of the plurality of laser pulses is no more than about 1 J/cm².

5. The system of claim 1, wherein a laser pulse of the plurality of laser pulses comprises a duration from about 1 to about 100 μsec.

6. The system of claim 1, further comprising a beam guidance system, wherein the beam guidance system is adapted to direct the plurality of laser pulses to respective tissue locations in a pattern.

7. The system of claim 6, wherein the pattern comprises a number of locations from about 15 locations to about 1500 locations.

8. The system of claim 6, wherein the beam guidance system is adapted to repeat directing the plurality of laser pulses to respective tissue locations in a pattern.

9. The system of claim 6, wherein the pattern comprises at least one tissue location, at least one location non-adjacent to the tissue location, and at least one location adjacent to the tissue location.

10. The system of claim 1, wherein the handpiece is adapted to form an exit orifice and operatively connected to the beam guidance system for delivering the laser beam to the hard treatment area.

11. The system of claim 10, wherein the handpiece further comprises a focusing optic and at least one optical lens, wherein the at least one optical lens is disposed between the beam guidance system and a tip.

12. The system of claim 11, wherein the at least one lens comprises two lenses.

13. The system of claim 11, wherein the focusing optic and the at least one lens are configured to increase a diameter of the laser beam.

14. The system of claim 11, wherein the focusing optic and the at least one lens are configured to generate a collimated laser beam.

15. A method for treating a treatment area of hard tissue, the method comprising the steps of:
generating a plurality of laser pulses of a laser beam having a wavelength from about 9 μm to about 10 μm using a CO₂ laser source; and
directing the plurality of laser pulses to respective tissue locations within a treatment area.
